(19) United States
(12) Patent Application Publication
(10) Pub. No.: US 2020/0044318 A1
(43) Pub. Date: Feb. 6, 2020

(54) Devices and Methods for the Delivery of Non-Thermal Therapeutic and Prophylactic Electromagnetic Fields from a Mobile Device

(71) Applicants: Andre' DIMINO, Woodcliff Lake, NJ (US); Iyer VISWANATHAN, Santa Clara, CA (US); Steven M. GLUCKSTERN, Santa Fe, NM (US); Endonovo Therapeutics, Inc., Woodland Hills, CA (US)

(72) Inventors: Arthur A. PILLA, Oakland, NJ (US); Andre' Dimino, Woodcliff Lake, NJ (US); Iyer Viswanathan, Santa Clara, CA (US); Steven M. Gluckstern, Santa Fe, NM (US)

(21) Appl. No.: 16/485,428

(22) PCT Filed: Feb. 23, 2018
(86) PCT No.: PCT/US2018/019457

§ 371 Date: Aug. 12, 2019

Related U.S. Application Data
(60) Provisional application No. 62/463,475, filed on Feb. 24, 2017.

(51) Int. Cl. H01Q 1/24; A61N 1/40; A61N 2/02

(52) U.S. Cl. CPC H01Q 1/244; A61N 2/02; A61N 1/40

(57) ABSTRACT

Systems for converting personal communications devices (e.g., smart phones) into devices for directly or wirelessly delivering either the native RF communications EMF signal or shaped and calibrated electromagnetic signals to promote cell and tissue growth, repair, and maintenance in plants, animals and humans which can modulate biochemical signaling pathways. In particular, described herein are systems, devices and methods for converting mobile communications devices (including PDAs, cell phones, mobile phones, etc.) to provide therapeutically-relevant EMF therapy (e.g., PEMF therapy), as well as methods of treating a patient using personal communications devices (PCDs) running an application for the delivery and monitoring of EMF therapy to a patient from the PCD.

### CROSS REFERENCE TO RELATED APPLICATIONS

This application may be related to any of the following patent applications, each of which is herein incorporated by reference in its entirety:

| | USPTO | Application | |
|---|---|---|---|
| Title | application No. | Filing Date | Issued/Date |
| APPARATUS AND METHOD FOR ELECTROMAGNETIC | 11/003,108 | Dec. 3, 2004 | 7,744,524 |
| TREATMENT OF PLANT, ANIMAL AND HUMAN TISSUE, | | | Issued on |
| ORGANS, CELLS AND MOLECULES | | | Jun. 29, 2010 |
| APPARATUS AND METHOD FOR ELECTROMAGNETIC | 12/771,954 | Apr. 30, 2010 | Now |
| TREATMENT OF PLANT, ANIMAL AND HUMAN TISSUE, | | | abandoned |
| ORGANS, CELLS AND MOLECULES | | | |
| APPARATUS AND METHOD FOR ELECTROMAGNETIC | 12/772,002 | Apr. 30, 2010 | Now |
| TREATMENT OF PLANT, ANIMAL AND HUMAN TISSUE, | | | abandoned |
| ORGANS, CELLS AND MOLECULES | | | |
| APPARATUS AND METHOD FOR ELECTROMAGNETIC | 12/819,956 | Jun. 21, 2010 | Now |
| TREATMENT | | | abandoned |
| ELECTROMAGNETIC TREATMENT INDUCTION APPARATUS | 11/114,666 | Apr. 26, 2005 | 7,740,574 |
| AND METHOD FOR USING SAME | | | issued on |
| | | | Jun. 22, 2010 |
| INTEGRATED COIL APPARATUS FOR THERAPEUTICALLY | 11/223,073 | Sep. 10, 2005 | 7,753,490 |
| TREATING HUMAN AND ANIMAL CELLS, TISSUES AND | | | issued on |
| ORGANS WITH ELECTROMAGNETIC FIELDS AND METHOD | | | Jul. 20, 2010 |
| FOR USING SAME | | | |
| ELECTROMAGNETIC FIELD TREATMENT APPARATUS AND | 12/082,944 | Apr. 14, 2008 | 7,896,797 |
| METHOD FOR USING SAME | | | issued on |
| | | | Mar. 11, 2011 |

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

This invention pertains generally to a method and apparatus for therapeutic and prophylactic treatment of animal and human molecules, cells, tissues, organs, portions of entire organisms and entire organisms. In particular, an embodiment according to the present invention pertains to use of time-varying electromagnetic fields to accelerate the asymmetrical kinetics of the binding of intracellular ions to their respective buffers which, in turn, activates the enzymes involved in the biochemical signaling pathways living systems employ for growth, repair and maintenance. Another embodiment according to the present invention pertains to application of sinusoidal, rectangular, chaotic or arbitrary waveform electromagnetic signals, having frequency components below about 1000 GHz, configured to accelerate the binding of intracellular Ca²⁺ to a buffer, such as calmodulin (hereinafter known as CaM), to enhance biochemical signaling pathways in target structures such as plant, animal and human molecules, cells, tissues, organs, portions of entire organisms and entire organisms. Signals configured according to embodiments of the present invention produce a net increase in a bound ion, such as Ca²⁺ at the binding sites of an intracellular buffer, such as CaM, because the asymmetrical kinetics of such binding allows such signals to cause the excess voltage induced at the ion binding site to accumulate, thereby accelerating voltage-dependent ion binding. A particular embodiment according to the present invention pertains to the non-thermal application of the repetitive pulse bursts of the native electromagnetic field signal from mobile communications devices (including PDAs, cell phones, mobile phones, etc.) to instantaneously accelerate ion-buffer binding in biochemical signaling pathways in structures such as molecules, cells, tissues, organs of animals or humans. Another particular embodiment according to the present invention is application of specific therapeutic electromagnetic fields by a modification of the circuitry of mobile communications devices (including PDAs, cell phones, mobile phones, etc.) to instantaneously accelerate ion-buffer binding in biochemical signaling pathways in structures such as molecules, cells, tissues, organs of animals or humans.

### BACKGROUND OF THE INVENTION

The application of weak non-thermal electromagnetic fields (“EMF”) can result in physiologically meaningful in vivo and in vitro bioeffects. Time-varying electromagnetic fields, comprising rectangular waveforms such as pulsing electromagnetic fields (“PEMF”), and sinusoidal waveforms such as pulsed radio frequency fields (“PRF”) ranging from several Hertz to an about 15 to an about 40 MHz range, are clinically beneficial when used as an adjunctive therapy for a variety of musculoskeletal injuries and conditions.

Beginning in the 1960's, development of modern therapeutic and prophylactic devices was stimulated by clinical problems associated with non-union and delayed union bone fractures. Early work showed that an electrical pathway can be a means through which bone adaptively responds to mechanical input. Early therapeutic devices used implanted and semi-invasive electrodes delivering direct current (“DC”) to a fracture site. Non-invasive technologies were subsequently developed using electrical and electromagnetic fields. These modalities were originally created to provide a non-invasive “no-touch” means of inducing an electrical/mechanical waveform at a cell/tissue level. Clinical applications of these technologies in orthopaedics have led to approved applications by regulatory bodies worldwide for treatment of fractures such as non-unions and fresh fractures, as well as spine fusion. Presently several EMF devices constitute the standard armamentarium of orthopaedic clinical practice for treatment of certain fresh and difficult to heal fractures. The success rate for these devices has been very high. The database for this indication is large enough to enable its recommended use as a safe, non-surgical, non-invasive alternative to a first bone graft for a delayed union fracture. Additional clinical indications for these original PEMF technologies have been reported in double blind studies for treatment of avascular necrosis, tendinitis, osteoarthritis, wound repair, blood circulation and pain from arthritis as well as other musculoskeletal injuries.

Cellular studies have addressed effects of weak electromagnetic fields on both signal transduction pathways and growth factor synthesis. It has been shown that EMF enhances the production of cytokines and growth factors after a short, trigger-like duration, lon/ligand binding processes at intracellular buffers attached to the cell membrane are generally accepted as an initial EMF target pathway structure. The clinical relevance to treatments for example of bone repair, is enhanced growth factor production staged within the normal biochemical cascades which regulate bone repair. Cellular level studies have shown effects on calcium ion transport, cell proliferation, Insulin Growth Factor (“IGF-II”) release, IGF-II and Adenosine receptor expression in osteoblasts. Effects on Insulin Growth Factor-I (“IGF-I”), IGF-II and Adenosine receptors have also been demonstrated in rat fracture callus. Stimulation of transforming growth factor beta (“TGF-β”) messenger RNA (“mRNA”) with PEMF in a bone induction model in a rat has been shown. Studies have also demonstrated upregulation of TGF-β mRNA by PEMF in human osteoblast-like cell line designated MG-63, wherein there were increases in TGF-β1, collagen, and osteocalcin synthesis. PEMF stimulated an increase in TGF-β1 in both hypertrophic and atrophic cells from human non-union tissue. Further studies demonstrated an increase in both TGF-β1 mRNA and protein in osteoblast cultures resulting from a direct effect of EMF on a calcium/calmodulin-dependent signaling pathway. Cartilage cell studies have shown similar increases in TGF-β1 mRNA and protein synthesis from EMF, demonstrating a therapeutic application to joint repair. U.S. Pat. No. 4,315,503 (1982) to Ryaby, U.S. Pat. No. 7,468,264 (2008) to Brighton and U.S. Pat. No. 5,723,001 (1998) and above referenced U.S. Pat. No. 7,744,524 (2010), both to Pilla, typify the research conducted in this field.

However, prior art in this field typically involves large, expensive, and difficult to use and control applicators. Such devices may apply unnecessarily high amplitude and power to a target pathway structure, require unnecessarily long treatment time, and are not portable or disposable. Thus, there is a need for an apparatus and a method that more effectively modulates biochemical processes that regulate tissue growth and repair, shortens treatment times, and incorporates miniaturized circuitry and light-weight applicators, thus allowing the apparatus to be portable, easy to use, and if desired disposable.

The invention(s) described herein proposes modification of the circuitry of a PDA to apply its native RF communication signals for prophylactic and therapeutic purposes. Therefore, the use of a cellular or smart phone or tablet to apply therapeutic and/or protective EMF/PEMF may provide an opportunity to make a completely portable EMF/PEMF device available to address these shortcomings, and may also provide additional advantages as noted below.

### SUMMARY OF THE DISCLOSURE

The present invention relates to mobile (e.g., hand-held) communications devices (e.g. cell phones, PDAs, iPads, iphones, smart phones, laptops, etc.) that are configured or adapted for the application of therapeutic electromagnetic signals to treat a patient, including the application of therapeutic pulsed electromagnetic fields (PEMF). Also described are software, firmware, and hardware to adapt a pre-existing mobile communications device (which may be referred to generically as “smart phones”) for the application of therapeutic EMF and/or PEMF. For example, described herein are applications that may be configured to run on a smartphone to apply EMF to a subject. In some variations an applicator (e.g., EMF/PEMF antenna) may be connected to the phone for the application of EMF/PEMF. The antenna may be a loop, and may include a connector (e.g., USB or other smart-phone compatible connector) that may be driven or controlled by the application software/firmware on the smartphone. In some variations the pre-existing RF signal and antenna of the smartphone may be adapted for the application of therapeutic EMF/PEMF.

In one embodiment, a combination electromagnetic treatment apparatus and wireless communication device used for treatment of animals and humans is provided having at least one waveform parameter that includes at least one of a frequency component parameter that configures said at least one waveform to repeat between about 0.01 Hz and about 100 MHz according to a mathematical function, a burst amplitude envelope parameter that follows a mathematically defined amplitude function, a burst width parameter that varies at each repetition according to a mathematically defined width function, a peak induced electric field parameter varying between about 1 μV/cm and about 100 mV/cm in said target pathway structure according to a mathematically defined function, and a peak induced magnetic electric field parameter varying between about 1 μT and about 0.1 T in said target pathway structure according to a mathematically defined function.

In general, the applicator may include a user interface, including instructions to the user/patient, controls (e.g., start/stop, power/intensity level settings, timer settings, mode settings, etc.) for setting or controlling the application of the therapeutic EMF, logging (for recording the sessions of use and information about the session, etc.). The application may also include control logic for controlling the application of EMF/PEMF to the subject using an applicator and/or the existing antenna loop present in the smartphone. The applicator may also include display logic for displaying one or more indicators that the energy (EMF/PEMF) is being applied, and/or one or more timers indicating the timing of the application or the time to the next application. In some variations the application may be software but may also include additional hardware that may be integrated with the device (e.g., within the smartphone housing) or it may be external and may plug into the smartphone (via one or more ports on the smartphone).

### BRIEF DESCRIPTION OF THE DRAWINGS

FIG. 1 is a schematic representation of a biological EMF transduction pathway which is a representative target pathway of the therapeutic or prophylactic EMF or PEMF signals that may be delivered from a smartphone as described in the present invention. According to the present invention, a therapeutic EMF/PEMF signal can accelerate cytosolic ion binding to a cytosolic buffer, such as Ca²⁺ binding to CaM, because the rate constant for binding, kₒₙ is voltage-dependent and much greater than the rate constant for unbinding (dissociation), k_{off}, imparting rectifier-like properties to ion-buffer binding, such as Ca²⁺ binding to CaM; cNOS* represents activated constitutive nitric oxide synthase (cNOS), catalyzes the production of NO from L-arginine; sGC* is activated guanylyl cyclase which catalyzes cyclic guanosine monophosphate (cGMP) formation when NO signaling modulates the tissue repair pathway; AC* is activated adenylyl cyclase, which catalyzes cyclic adenosine monophosphate (cAMP) when NO signaling modulates differentiation and survival.

FIG. 2A is a schematic diagram showing one variation of a personal communications device (PCD) including an application (“driver”) for controlling application of therapeutic EMF/PEMF to a subject; in this variation the applicator uses the internal antenna (“applicator”) within the phone to apply the therapeutic energy to the subject. FIG. 2A also illustrates modification of a cellular phone to apply its native communications signals for prophylactic and therapeutic purposes.

FIG. 2B is a schematic of a second variation of a PCD including an application (“driver”) for controlling application of therapeutic EMF/PEMF, including the native communications signal to a subject; in this variation the applicator uses an external applicator that plugs into the PCD for application of the EMF/PEMF.

FIG. 3A is another example of a PCD housing (in this example, a smartphone housing) configured to run an application that adapts the smartphone into a therapeutic EMF therapy device.

FIG. 3B is another example of a smartphone configured to run an application that adapts the smartphone into a therapeutic EMF therapy device.

FIG. 4 is an example of a PCD (smartphone) running an application to drive the delivery of therapeutic EMF to a patient from the connected applicator loop.

FIG. 5 schematically illustrates one variation of a system including a PCD and PEMF applicator.

### DETAILED DESCRIPTION OF THE PREFERRED EMBODIMENTS

Basal levels of certain intracellular ions are tightly maintained by a number of physiological calcium buffers. It is generally accepted that transient elevations in cytosolic, e.g., Ca²⁺ from external stimuli as simple as changes in temperature and mechanical forces, or as complex as mechanical disruption of tissue, rapidly activate CaM, which equally rapidly activates the cNOS enzymes, i.e., endothelial and neuronal NOS, or eNOS and nNOS, respectively. Studies have shown that both isoforms are inactive at basal intracellular levels of Ca²⁺, however, their activity increases with elevated Ca²⁺. Thus, nNOS and eNOS are regulated by changes in intracellular Ca²⁺ concentrations within the physiological range. In contrast, a third, inducible isoform of NOS (iNOS), which is upregulated during inflammation by macrophages and/or neutrophils, contains CaM that is tightly bound, even at low resting levels of cytosolic Ca²⁺, and is not sensitive to intracellular Ca²⁺.

Once cNOS is activated by CaM it converts its substrate, L-arginine, to citrulline, releasing one molecule of NO. As a gaseous free radical with a half-life of about 5 sec, NO diffuses locally through membranes and organelles and acts on molecular targets at a distance up to about 200 μm. The low transient concentrations of NO from cNOS can activate soluble guanylyl cyclase (sGC), which catalyzes the synthesis of cyclic guanosine monophosphate (cGMP). The CaM/NO/cGMP signaling pathway is a rapid response cascade which can modulate peripheral and cardiac blood flow in response to normal physiologic demands, as well as to inflammation. This same pathway also modulates the release of cytokines, such as interleukin-1beta (IL-1β) and growth factors such as basic fibroblast growth factor (FGF-2) and vascular endothelial growth factor (VEGF) which have pleiotropic effects on cells involved in tissue repair and maintenance.

Following an injury, e.g., a bone fracture, torn rotator cuff, sprain, strain or surgical incision, repair commences with an inflammatory stage during which the pro-inflammatory cytokine IL-1β is rapidly released. This, in turn, up-regulates iNOS, resulting in the production of large amounts of NO in the wound bed. Continued exposure to NO leads to the induction of cyclooxygenase-2 and increased synthesis of prostaglandins which also play a role in the inflammatory phase. While this process is a natural component of healing, when protracted, it can lead to increased pain and delayed or abnormal healing. In contrast, CaM/eNOS/NO signaling has been shown to attenuate levels of IL-1β and down-regulate iNOS. As tissue further responds to injury, the CaM/NO/cGMP cascade is activated in endothelial cells to stimulate angiogenesis, without which new tissue growth cannot be sustained.

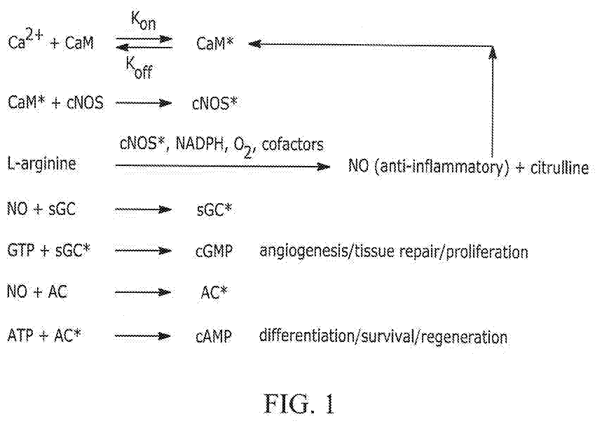

One EMF transduction pathway relevant to tissue maintenance, repair and regeneration, begins with voltage-dependent Ca²⁺ binding to CaM. Ca/CaM binding produces activated CaM which binds to, and activates, cNOS, which catalyzes the synthesis of the signaling molecule NO from L-arginine. This pathway is shown in its simplest schematic form in FIG. 1. According to the present invention, EMF can accelerate the kinetics of Ca²⁺ binding to CaM, the first step of a well characterized cascade that responds to stress and chemical or physical insults. Ca/CaM binding is kinetically asymmetrical, i.e., the rate of binding exceeds the rate of dissociation by several orders of magnitude (kₒₙ>>k_{off}), driving the reaction in the forward direction. Ca/CaM binding has been well characterized, with the binding time constant reported to be in the range of 10⁻²-10⁻³ sec. In contrast, release of Ca²⁺ from CaM cannot occur until cNOS* has converted L-arginine to citrulline and NO, which takes the better part of a second. Subsequent reactions involving NO depend upon the cell/tissue state. For example, tissue repair requires a temporal sequence of inflammatory, anti-inflammatory, angiogenic and proliferative components. Endothelial cells orchetrate the production of FGF-2 and VEGF for angiogenesis. For each of these phases, early NO production by endothelial cells, leading to increased cGMP by these, as well as other NO targets, such as vascular smooth muscle, would be expected to be modulated by an EMF effect on sGC via Ca/CaM binding. In contrast, nerve or bone regeneration may require other pathways leading to differentiation during development and growth, and prevention of apoptosis, as in response to injury or neurodegenerative diseases. For these cases, early cyclic adenosine monophosphate (cAMP) formation would be modulated by an EMF effect on sAC via Ca/CaM binding.

The substantial asymmetry of Ca/CaM binding kinetics provides a unique opportunity for the communication signals from smartphones to selectively modulate kon. In general, if kₒₙ>>k_{off}, and kₒₙ is voltage-dependent, according to the present invention, ion binding could be increased with an exogenous bipolar electric field signal having a carrier period or pulse duration that is significantly shorter than the mean lifetime of the bound ion. This applies to the CaM signaling pathway, causing it to exhibit rectifier-like properties, i.e., to yield a net increase in the population of bound Ca²⁺ because the forward (binding) reaction is favored. The change in surface concentration, ΔΓ, of Ca²⁺ at CaM is equal to the net increase in the number of ions that exit the outer Helmholtz plane, penetrate the water dipole layer at the aqueous interface of the binding site, and become bound in the inner Helmoltz plane. For the general case of ion binding, evaluation of Ca/CaM binding impedance, ZA(s), allows calculation of the efficacy of any given waveform in that pathway by evaluating the frequency range over which the forward binding reaction can be accelerated. Thus, binding current, IA(t), is proportional to the change in surface charge (bound ion concentration) via dq(t)/dt, or, in the frequency domain, via sqA(s). IA(s) is, thus, given by:

I_{A}(s)=sq_{A}(s)=sΓₒf(ΔΓ(s)) (1)

where s is the real-valued frequency variable of the Laplace transform. Taking the first term of the Taylor expansion of equation 1 gives:

I_{A}(s)=q_{Γ}sΓₒΔΓ(s) (2)

where q_{Γ}=∂q/∂Γ, a coefficient representing the dependence of surface charge on bound ion concentration. ΔΓ(s) is a function of the applied voltage waveform, E(s), and, referring to the reaction scheme in FIG. 1, of the change in concentration of eNOS*, defined as ΔΦ(s):

ΔΓ(s)=kₒₙ/Γₒs[−ΔΓ(s)+aE(s)+ΔΦ(s)] (3)

where Γₒ is the initial surface concentration of Ca2+(homeostasis), and a=∂Γ/∂E, representing the voltage dependence of Ca2+ binding. Referring to the reaction scheme in FIG. 1, it may also be seen that eNOS* depends only upon Ca2+ binding, i.e., ΔΓ(s). Thus:

ΔΦ(s)=υ_{Φ}/Φₒs[−ΔΦ(s)−ΔΓ(s)] (4)

where υΦ is the rate constant for Ca/CaM binding to eNOS and Φo is the initial concentration of eNOS* (homeostasis).
Using equations 2, 3 and 4, and for kₒₙ>>υ_{Φ}, ZA(s) may be written:

$${Z}_{A} ( s ) = \frac{E ( s )}{{I}_{A} ( s )} = \frac{1}{{q}_{r} a} [ \frac{1 + {Γ}_{o} s \text{/} {k}_{on}}{{Γ}_{o} s} ] \qquad ( 5 )$$

Equation (5) describes the overall frequency response of the first binding step in a multistep ion binding process at an electrified interface, wherein the second step requires that the bound ion remain bound for a period of time significantly longer than the initial binding step. For this case, the first ion binding step is represented by an equivalent electrical impedance which is functionally equivalent to that of a series R_{A}-C_{A} electric circuit, embedded in the overall dielectric properties of the target. R_{A} is inversely proportional to the binding rate constant (kₒₙ), and C_{A} is directly proportional to bound ion concentration.

The present invention teaches that a bipolar electromagnetic field, for which pulse duration or carrier period is less than about half of the bound ion lifetime can be configured to maximize current flow into the capacitance C_{A}, which will increase the voltage, E_{b}(s), where s is the Laplace frequency, across C_{A}. E_{b}(s) is a measure of the increase in the surface concentration of the binding ion in the binding sites of the buffer, above that which occurs naturally in response to a given physiological state. The result is an increase in the rate of biochemical signaling in plant, animal and human repair, growth and maintenance pathways which results in the acceleration of the normal physiological response to chemical or physical stimuli. The following equation demonstrates the relation between the configured electromagnetic waveform, E(s) and E_{b}(s).

$${E}_{b} ( s ) = \frac{( 1 \text{/} {sC}_{A} ) E ( s )}{{( {R}_{A}^{2} + {( 1 \text{/} {sC}_{A} )}^{2} )}^{1 / 2}} \qquad ( 6 )$$

The present invention further teaches that a time-varying bipolar electromagnetic field for which pulse duration or carrier period is less than about half of the bound ion lifetime of Ca²⁺ binding to CaM will maximize the current flow into the Ca/CaM binding pathway to accelerate the CaM-dependent signaling which plants, animals and humans utilize for tissue growth, repair and maintenance. In particular, a time-varying bipolar electromagnetic field may be configured to modulate CaM-dependent NO/cGMP signaling which accelerates; pain and edema relief, angiogenesis, hard and soft tissue repair, repair of ischemic tissue, prevention and repair of neurodegenerative diseases, nerve repair and regeneration, skeletal and cardiac muscle repair and regeneration, relief of muscle pain, relief of nerve pain, relief of angina, relief of degenerative joint disease pain, healing of degenerative joint disease, immunological response to disease, including cancer. In addition, prophylactic application of such signals can provide a protective effect to any tissue or organ in the living system in anticipation of stress or physical or chemical insult.

It has been shown that quiescent cells and tissues can respond to EMF by producing heat shock proteins (HSP). These studies showed that tissue protection is related to an increase, by EMF, in the release of HSP70. This is consistent with the use of EMF for signaling. Thus, because ion binding is voltage-dependent and kinetically asymmetrical, EMF signals, can be configured to drive the forward (binding) reaction and activate the buffer even when cytosolic ion concentrations are at baseline (tissue at rest). The physiological import of this stems from evidence that NO induces the expression of HSP. It follows that HSP expression can be enhanced via an EMF effect on CaM-dependent NO signaling, even for quiescent cells and tissues. Release of HSP prior to injury can reduce the inflammatory response because it is poised to rapidly downregulate IL-1β and iNOS, which would protect tissue from injury.

Utilization of the above analysis on the RF communication signals emitted by smartphones shows that these signals satisfy the requirements to affect, e.g., Ca²⁺ binding to CaM. Therefore, a preferred embodiment according to the present invention is modification of the software of a smartphone, activated by a switch, to apply predefined periods of the RF communication signal to target structures such as plant, animal and human molecules, cells, tissues, organs, portions of entire organisms and entire organisms for therapeutic and prophylactic purposes.

FIG. 1 schematically illustrates examples of the biochemical signaling pathways which can be modulated by acceleration of Ca²⁺ binding to CaM with non-thermal electromagnetic fields (EMF) configured according to the present invention are illustrated. The application of non-thermal EMF, configured according to the present invention, instantaneously accelerates the kinetics of Ca²⁺ binding to CaM, the first step of a well characterized signaling cascade which a plant, animal or human organism utilizes to respond to chemical or physical insults. Ca/CaM binding is kinetically asymmetrical, i.e., the rate of binding exceeds the rate of dissociation by several orders of magnitude (kₒₙ>>k_{off}), therefore the application of EMF will instantaneously drive the reaction in the forward direction. The Ca/CaM binding time constant is in the range of 1 to 10 milliseconds. In contrast, the release of Ca²⁺ from CaM cannot occur until cNOS* has converted L-arginine to citrulline and NO, which takes the better part of a second. Subsequent reactions involving NO depend upon the cell/tissue state. For example, tissue repair requires a temporal sequence of inflammatory, anti-inflammatory, angiogenic and proliferative components. Endothelial cells orchestrate the production of FGF-2 and VEGF for angiogenesis. For each of these phases, early NO production by endothelial cells, leading to increased cGMP by these, as well as other NO targets, such as vascular smooth muscle, are modulated by an EMF effect on GC via Ca/CaM binding. In contrast, nerve or bone regeneration require other pathways leading to differentiation during development and growth, and prevention of apoptosis, as in response to injury or neurodegenerative diseases. For these cases, early cAMP formation is modulated by an EMF effect on AC via Ca/CaM binding.

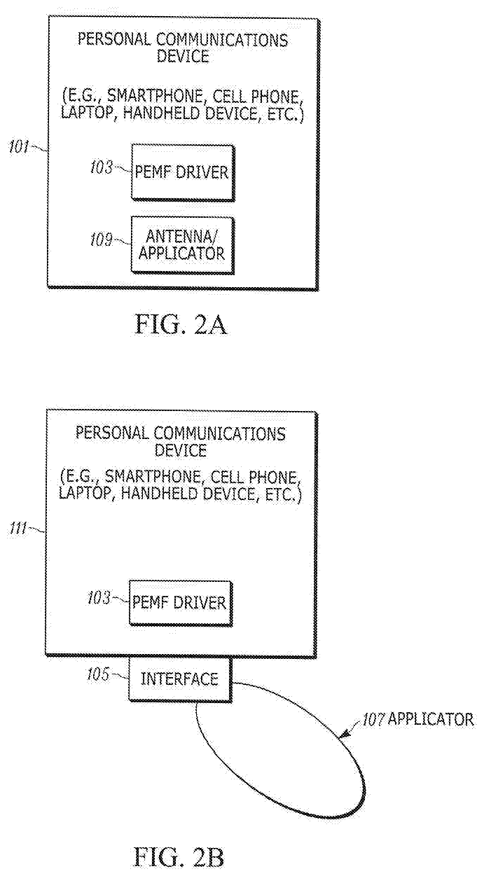

FIGS. 2A and 2B schematically illustrate two variations of smart phones 101 and 11, respectively, that are configured to run an application adapting the smartphone to operate as a PEMF applicator. For example, in FIG. 2A, the personal communication device (PCD) 101 is shown running an application (“driver” 103) that adapts the PCD 101, in this case a phone 101, to apply regimens of its RF communications signal as therapeutic or prophylactic EMF. In this example, timing circuitry, placed into operation with a switch (neither shown in FIG. 2A), drives the internal antenna 109 (e.g., a loop antenna) within the shell of the PCD 101 to emit its RF communication signal for a predefined time that is therapeutically and/or prophylactically relevant.

In general, the PCD may be any appropriate communications device, including smart phones (e.g., iPhones™, Droids™, etc.), laptop/palmtop computers (e.g., iPad™, etc.), or any other mobile communications device having sufficient processing power to run the application “driver” 103. In FIG. 2A, the PCD 101 includes an internal antenna 109 that may act as an applicator for the application of its RF communications EMF signal. In this example the driver 103 may be software, firmware and/or hardware, and may be uploaded on the PCD, or pre-loaded onto the PCD. The driver 103 may control the application of EMF/PEMF by the internal applicator/antenna 109. The native RF communications signal may be emitted using the power supply for the PCD (e.g., battery, etc.), or it may be provided by a separate module that may be connected to the PCD (e.g., using a port on the PCD).

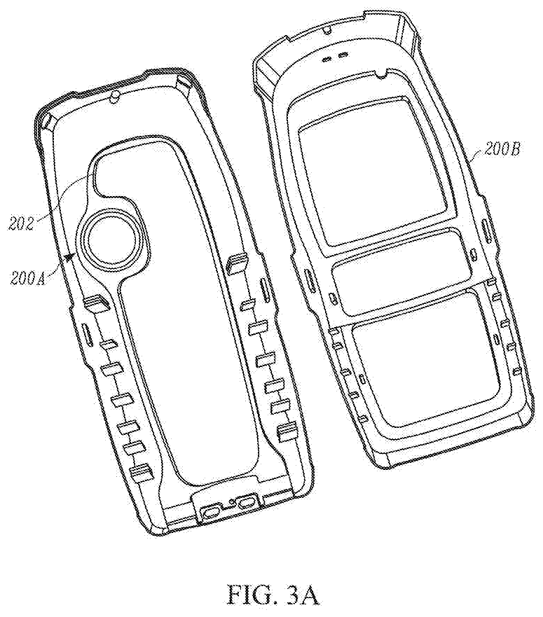

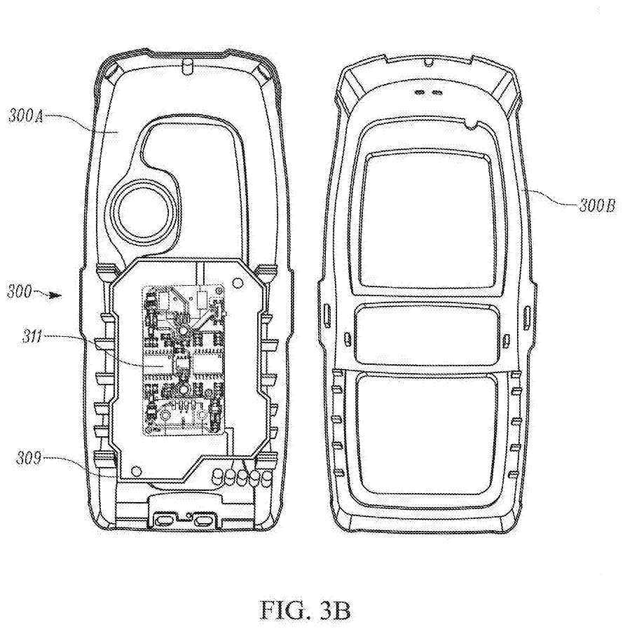

FIGS. 3A and 3B illustrate a similar variation of a smartphone housing 200, comprising cooperating shells 200A and 200B, and including an internal antenna (applicator loop) and control circuit that is located inside the phone housing 200, which is shown exposed. An inductive loop 202 is shown disposed adjacent the inner surface of one of the housing shells 200A that may be used as an applicator to apply EMF controlled by a driver, similar to driver 103 of FIG. 1, running on the smartphone which allows it to emit any other EMF/PEMF signal found to be therapeutically or prophylactically relevant.

FIG. 3B shows another variation of a smart phone housing 300, comprising cooperating shells 300A and 300B, as well as appropriate circuitry 311 capable of running an application to deliver EMF therapy from an internal driver on the smartphone 300. The internal loop antenna 309 is shown arrayed around and adjacent the inner surface of one of the smartphone housing shells 300A.

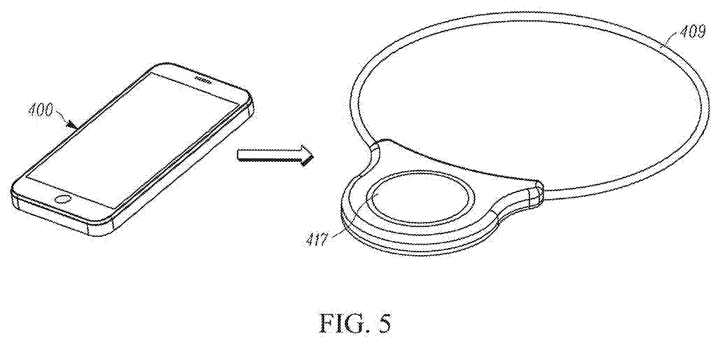

Returning now to FIG. 2B, in some variations the system 111 may include an external applicator (e.g., loop) 107 to deliver EMF to a patient/subject by running an application (driver) 103 on the PCD when the applicator is connected. In some variations, the applicator includes an extension 115 that plugs into a port 125 (FIG. 5) on the PCD 111 (e.g., a USB port, etc.). The external applicator may be merely an antenna 107 (e.g., loop; FIG. 2B), or it may include additional hardware and/or software that is driven by the driver application. e.g., driver 103, running on the PCD 111. For example, the system may include an interface 105 between the applicator 107 and the driver 103; the interface 105 may include one or more amplifiers (not shown) for driving the EMF/PEMF to be emitted by the loop 107 at an optimal therapeutic level. In some variations, the interface includes a port adapter extension 115 (FIG. 5) configured to connect the applicator 107 to an available port, (e.g., port 115, FIG. 5) disposed at and edge of the PCD 11. The interface may also include hardware/software/firmware to help drive the appropriate level of EMF from the applicator 107.

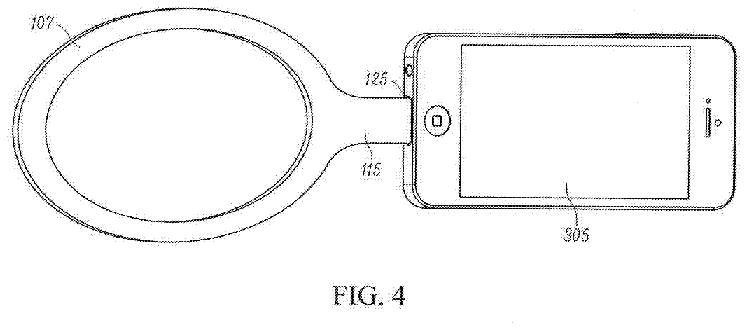

FIG. 4 shows another variation of a PCD 300 (in this example, shown as a smartphone or iphone) having an internal applicator, such as loop antenna 309, and running application software to drive the delivery of therapeutic EMF from the loop 309.

The applicator, either internal or external, may be flexible (e.g., made of cable, wire, etc.) or it may be substantially stiff or rigid.

FIG. 5 shows yet another variation of a PCD 400 which is used to wirelessly connect to an external coil antenna 409 and thereby to control the PEMF signal from the PCD 400. Appropriate wireless communications hardware, such as receiver 417, is electronically connected to the loop antenna 409 to apply any EMF/PEMF signal found to be therapeutically or prophylactically relevant, including the native RF communication signal, and to wirelessly monitor the delivery of such EMF/PEMF signals to the plant, animal and human molecules, cells, tissues, organs, portions of entire organisms and entire organisms where therapeutic or prophylactic EMF/PEMF action is desired.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes, modifications, alterations and variations may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A combination electromagnetic treatment apparatus and mobile communication device used for treatment of animals and humans comprising:
a mobile communication device;
a waveform configuration means in electronic communication with the mobile communication device for configuring at least one waveform to be coupled to a target pathway structure according to a mathematical model having at least one waveform parameter capable of being chosen to satisfy a Signal to Noise Ratio model so that said at least one waveform is configured to be detectable in said target structure above background activity in said target pathway structure;
at least one coupling device connected to said waveform configuration means by at least one connecting means for generating an electromagnetic signal from said configured at least one waveform and for coupling said electromagnetic signal to said target pathway structure; and
a positioning device wherein said at least one coupling device is integral to said positioning device.

2. The combination electromagnetic treatment apparatus and mobile communication device used for treatment of animals and humans according to claim 1, wherein the waveform configuration means is capable of generating an electromagnetic signal, wherein the electromagnetic signal further comprises a bipolar electromagnetic field having a pulse duration less than about half of a bound ion lifetime, and is defined by the following equation
$${E}_{b} ( s ) = \frac{( 1 \text{/} {sC}_{A} ) E ( s )}{{( {R}_{A}^{2} + {( 1 \text{/} {sC}_{A} )}^{2} )}^{1 / 2}} \qquad$$
where E_{b}(s) is a measure of the increase in the surface concentration of the binding ion in the binding sites of the buffer, above that which occurs naturally in response to a given physiological state and the relation between the configured electromagnetic waveform, E(s) and E_{b}(s)
C_{A} is the nominal capacitance of a first ion binding step as represented by an equivalent electrical impedance which is functionally equivalent to that of a series R_{A}-C_{A} electric circuit, embedded in the overall dielectric properties of the target tissue of a human or animal;
R_{A} is inversely proportional to the binding rate constant (kₒₙ);
C_{A} is directly proportional to bound ion concentration
s is the Laplace frequency across C_{A};
whereby the electromagnetic field can be configured to maximize current flow into a the maximize current flow into the capacitance C_{A}, thereby increasing the voltage Eb(s).

3. The combination electromagnetic treatment apparatus and mobile communication device used for treatment of animals and humans according to claim 1, wherein the mobile communication device further comprises a PDA.

4. The combination electromagnetic treatment apparatus and mobile communication device used for treatment of animals and humans according to claim 1, wherein the mobile communication device further comprises a cell phone.

5. The combination electromagnetic treatment apparatus and mobile communication device used for treatment of animals and humans according to claim 1, wherein the mobile communication device further comprises a mobile phone.

6. The combination electromagnetic treatment apparatus and mobile communication device used for treatment of animals and humans according to claim 1, wherein the mobile communication device further comprises a loop antenna configured for delivery of the electromagnetic field to the of the target tissue of the animal or human.

7. The combination electromagnetic treatment apparatus and mobile communication device used for treatment of animals and humans according to claim 6, wherein the loop antenna is disposed internally within a housing of the mobile communication device.

8. The combination electromagnetic treatment apparatus and mobile communication device used for treatment of animals and humans according to claim 6, wherein the loop antenna is disposed externally of the mobile communication device.

9. The combination electromagnetic treatment apparatus and mobile communication device used for treatment of animals and humans according to claim 8, wherein the externally disposed loop antenna further comprises a means for electronic communication with the waveform configuration means capable of generating an electromagnetic signal disposed within the mobile communication device.

10. The combination electromagnetic treatment apparatus and mobile communication device used for treatment of animals and humans according to claim 8, wherein the externally disposed loop antenna further comprises a means for wireless electronic communication with the waveform configuration means capable of generating an electromagnetic signal disposed within mobile communication device.

11. The combination electromagnetic treatment apparatus and mobile communication device used for treatment of animals and humans according to claim 8, wherein the externally disposed loop antenna further comprises a means for attaching to a port on the mobile communication device thereby proving communication to the waveform configuration means capable of generating an electromagnetic signal disposed within mobile communication device.

12. The combination electromagnetic treatment apparatus and mobile communication device used for treatment of animals and humans according to claim 1, wherein the mobile communications device further includes one of software, firmware, and hardware configured to adapt the mobile communications device for the application of therapeutic EMF and/or PEMF.

13. The combination electromagnetic treatment apparatus and mobile communication device used for treatment of animals and humans according to claim 1, wherein the mobile communications devices further includes one of software, firmware and hardware configured to provide the at least one waveform parameter, the at least one waveform parameter including at least one of a frequency component parameter that configures said at least one waveform to repeat between about 0.01 Hz and about 100 MHz according to a mathematical function, a burst amplitude envelope parameter that follows a mathematically defined amplitude function, a burst width parameter that varies at each repetition according to a mathematically defined width function, a peak induced electric field parameter varying between about 1 μV/cm and about 100 mV/cm in said target pathway structure according to a mathematically defined function, and a peak induced magnetic electric field parameter varying between about 1 μT and about 0.1 T in said target pathway structure according to a mathematically defined function.

14. The combination electromagnetic treatment apparatus and mobile communication device used for treatment of animals and humans according to claim 13, wherein the mobile communication device further comprises a PDA.

15. The combination electromagnetic treatment apparatus and mobile communication device used for treatment of animals and humans according to claim 13, wherein the mobile communication device further comprises a cell phone.

16. The combination electromagnetic treatment apparatus and mobile communication device used for treatment of animals and humans according to claim 13, wherein the mobile communication device further comprises a mobile phone.

17. The combination electromagnetic treatment apparatus and mobile communication device used for treatment of animals and humans according to claim 13, wherein the mobile communication device further comprises a loop antenna configured for delivery of the electromagnetic field (EMF and/or PEMF) to the of the target tissue of the animal or human.

18. The combination electromagnetic treatment apparatus and mobile communication device used for treatment of animals and humans according to claim 17, wherein the loop antenna is disposed internally within a housing of the mobile communication device.

19. The combination electromagnetic treatment apparatus and mobile communication device used for treatment of animals and humans according to claim 17, wherein the loop antenna is disposed externally of the mobile communication device.

20. The combination electromagnetic treatment apparatus and mobile communication device used for treatment of animals and humans according to claim 19, wherein the externally disposed loop antenna further comprises a means for electronic communication with the waveform configuration means capable of generating an electromagnetic signal disposed within the mobile communication device.

21. The combination electromagnetic treatment apparatus and mobile communication device used for treatment of animals and humans according to claim 19, wherein the externally disposed loop antenna further comprises a means for wireless electronic communication with the waveform configuration means capable of generating an electromagnetic signal disposed within mobile communication device.

22. The combination electromagnetic treatment apparatus and mobile communication device used for treatment of animals and humans according to claim 19, wherein the externally disposed loop antenna further comprises a means for attaching to a port on the mobile communication device thereby proving communication to the waveform configuration means capable of generating an electromagnetic signal disposed within mobile communication device.
